(19) United States
(12) Patent Application Publication
(10) Pub. No.: US 2026/0000210 A1
(43) Pub. Date: Jan. 1, 2026

(54) HOLISTIC AND THERAPEUTIC FURNITURE AND METHODS OF MAKING THE SAME

(71) Applicant: Line29, Inc., San Diego, CA (US)

(72) Inventor: Casey Browar, San Diego, CA (US)

(21) Appl. No.: 18/915,026

(22) Filed: Oct. 14, 2024

Related U.S. Application Data
(60) Provisional application No. 63/665,094, filed on Jun. 27, 2024.

(51) Int. Cl. A47C 17/86; A47C 7/24; A47C 17/02; A61M 21/00; A61M 21/02

(52) U.S. Cl. CPC A47C 17/86; A47C 7/24; A47C 17/02; A61M 21/02; A61M 2021/0077; A61M 2205/0205

(57) ABSTRACT

The furniture described consists essentially of a structural member made of wood and incorporates high-frequency metals, including a selection from copper, silver, gold, zinc, magnesium, titanium, and platinum. Upholstered in a high-frequency fabric, options for this upholstery include cotton, silk, linen, wool, bamboo, hemp, or Tencel (Lyocell). Inside the furniture, multiple high-frequency crystals are strategically placed within internal compartments. In addition, copper balls may be positioned in close proximity to the high-frequency crystals to amplify their high-frequency affects.

### CROSS-REFERENCE TO RELATED APPLICATIONS

This application claims priority to U.S. Provisional Patent Application 63/665,094, filed Jun. 27, 2024, the entire contents of which are fully incorporated herein with this reference.

### FIELD

The present patent document relates to furniture. In particular, the present patent document relates to furniture that can provide therapeutic benefits and methods of making the same.

### BACKGROUND

In today's fast-paced society, where stress and imbalance are prevalent, many individuals are turning to alternative methods to promote holistic wellness and harmony. Holistic practices, rooted in the interconnectedness of the mind, body, and spirit, offer avenues for achieving well-being beyond conventional approaches. Central to these practices is the utilization of materials that provide unique energetic properties believed to aid in restoring balance and vitality.

Holistic wellness embraces the integration of physical, emotional, and spiritual aspects of health, acknowledging their profound interdependence in achieving overall well-being

In ancient holistic traditions like Ayurveda and traditional Chinese medicine, the concept of chakras, or energy centers, is foundational. It is believed that imbalances in these energy centers can manifest as physical or emotional ailments. Certain materials are thought to resonate with different chakras, each possessing unique vibrational frequencies capable of harmonizing and aligning these energy centers. This alignment is believed to facilitate the free flow of energy throughout the body, promoting overall wellness.

Furthermore, holistic practices often emphasize the significance of vibrational frequencies in influencing health. It is believed that everything, including our bodies and natural materials, emits vibrations at varying frequencies. Higher frequencies are associated with states of improved health and well-being, while lower frequencies may contribute to imbalance and discomfort.

By incorporating materials with higher vibrational frequencies individuals aim to elevate their energetic state and promote optimal health and vitality.

In essence, holistic wellness practices offer alternative pathways to well-being, emphasizing the interconnectedness of various dimensions of the self. By integrating certain natural materials into their surroundings, individuals tap into the innate healing power of nature, fostering harmony within themselves and their surroundings, while also aligning with higher frequencies conducive to holistic wellness.

In today's society, individuals spend a significant portion of their lives sitting in chairs, sitting or lying on couches, lying in beds or in close proximity to any number of other types of furniture. From the corporate office to the home, people spend significant portions of their life interfacing and/or interacting with furniture. To this end, there is a need in the market to create furniture that can not only function as furniture but to also provide holistic and therapeutic health benefits to anyone using the furniture. There is a need in the market for furniture design to take advantage of the chakars, known healing frequencies and/or sacred geometries.

### SUMMARY OF THE EMBODIMENTS

The preferred embodiments of the products and methods disclosed herein use materials known to carry high energy frequencies and that are used in Ayurvedic and traditional Chinese medicine as a holistic way to support physical, emotional, and energetic wellbeing.

Incorporating frequencies into the furniture through carefully selected materials and placement of those materials can help to foster deeper grounding connection with nature and promote harmony between the mind, body and spirit.

The combination of copper and the powerful healing energy from crystals creates a grounding and calming experience, softening the energy of the day. The synergistic effects of these materials may amplify their health benefits, offering users a comprehensive wellness experience within their living spaces.

The furniture described in the embodiments herein consists essential of high frequency materials. Natural wood, is a core component and may be used for the structural members. Preferably, this wood is pine wood but other woods may be used. The furniture incorporates high-frequency metals, including a selection from copper, aluminum, brass, tin, iron, bronze, silver, gold, zinc, magnesium, titanium, and platinum. In other embodiments, the furniture incorporates high-frequency metals including a selection from copper, aluminum, silver, gold, brass, tin, zinc and iron.

The upholstery used in this furniture is crafted from high-frequency fabric. In most embodiments, between 14 and 22 yards of high frequency material may be used. Of course, other embodiments, such as an end table or other table may use way less and pieces such as sectional couches may use more. High-frequency fabric may be chosen from a variety of high-frequency materials, such as 100% Linen, or 100% wool. However, as explained in more detail herein, wool and linen should not be both used on the same piece of furniture, as it would result in zero frequency.

Inside the furniture, there are multiple high-frequency crystals placed in internal compartments. These crystals can range between one and ten pounds in total weight. For optimal performance, a plurality of copper balls is positioned in close proximity to the high-frequency crystals. The copper may amplify the effects of the crystals. In some embodiments, other forms of copper, such as copper mesh lined pockets, may be used to get the same amplification affect as copper balls.

The specific types of high-frequency crystals incorporated into the furniture include options such as red jasper, hematite, amethyst, rose quartz, clear quartz, citrine, yellow jasper, and rose quartz, either alone or in combination. Notably, some configurations feature amethyst and rose quartz crystals together. Additionally, the high-frequency crystals may be enclosed in copper mesh fabric pockets or may be foiled in copper.

This unique combination of materials and design elements culminate in a piece of furniture, such as a sofa, that harnesses the benefits of high-frequency components for enhanced functionality and aesthetic appeal.

### BRIEF DESCRIPTION OF THE DRAWINGS

FIG. 1 illustrates an embodiment of a holistic and therapeutic sofa designed using the principles taught herein;

FIG. 2 illustrates the location of different Chakras on the human body;

FIG. 3 illustrates an embodiment of a holistic and therapeutic sofa similar to the one in FIG. 1 but with an additional copper back;

FIG. 4A illustrates a top-down plan view of the sofa of FIGS. 1 and 3 with some exemplary dimensions for reference.

FIG. 4B illustrates a front elevation view of the sofa of FIGS. 1 and 3 with some exemplary dimensions for reference.

FIG. 4C illustrates a rear elevation view of the sofa of FIGS. 1 and 3 with some exemplary dimensions for reference.

FIG. 4D illustrates a left-side elevation view of the sofa of FIGS. 1 and 3 with some exemplary dimensions for reference.

FIG. 4F illustrates a right-side elevation view of the sofa of FIGS. 1 and 3 with some exemplary dimensions for reference.

FIG. 5A illustrates a top-down plan view of the interior of the sofa of FIGS. 1 and 3.

FIG. 5B illustrates a front elevation view of the interior of the sofa of FIGS. 1 and 3.

FIG. 6 illustrates an internal view of one embodiment of a sofa made according to the teachings herein.

### DETAILED DESCRIPTION OF THE DRAWINGS

In preferred embodiments of the furniture disclosed herein, high-frequency materials are preferably used. High-frequency materials may aid in grounding, healing and overall wellbeing. In some embodiments, the furniture described herein differs from other furniture in that it consists essentially of only high-frequency materials. Accordingly, the materials used in the furniture is limited to high-frequency components and those that do not materially affect the basic and novel characteristics of the furniture. In such embodiments, only high-frequency materials are used for all the major components and fasteners are the only components that are not high-frequency components. In yet other embodiments, only high-frequency materials are used for all the major components and fasteners and filling/stuffing or batting for the cushions are the only components that are not high-frequency components. In still yet other embodiments, only high-frequency materials are used for all the major components including fasteners and only filling/stuffing/batting in the cushions is made from materials that are not high-frequency components. In still yet other embodiments, all of the components in the furniture, including the fasteners and stuffing/filling/batting are made from high-frequency materials. In such embodiments, the furniture is consisting of only high frequency materials.

There are a few metals that are considered to be high-frequency materials. One metal that is a high-frequency material is copper. Accordingly, in some embodiments, copper is used in the construction of the furniture. Copper is known to promote vitality and conductivity. Copper is renowned for its anti-inflammatory and antimicrobial properties. Studies have demonstrated that copper surfaces can effectively eliminate a wide range of pathogens, contributing to a cleaner, healthier environment. Copper is believed to resonate the body's electromagnetic field, promoting vitality and energy flow. Copper is also a strong conductor and amplifier.

Copper may be used on the furniture in a decorative manner, structural component or simply added within the furniture for its healing powers or any combination thereof.

In addition to Copper, the following high-frequency metals are often referenced in holistic health practices for their potential healing effects as may be considered high-frequency materials as taught herein: Silver, Gold, Zinc, Magnesium, Titanium, and Platinum. Each of these metals is believed to contribute uniquely to health and wellness through various mechanisms.

Silver is recognized for its antibacterial and antimicrobial properties. It has been utilized in various forms, including colloidal silver, believed to confer health benefits by inhibiting the growth of harmful microorganisms. This metal is often integrated into health products aimed at enhancing the body's ability to resist infections.

Gold is associated with promoting healing and reducing inflammation, making it a valuable element in holistic health approaches. It is believed to balance energy within the body and is often employed in alternative therapies through topical applications and supplements designed to support overall wellness and vitality.

Zinc plays a critical role in immune function and wound healing. It is widely recognized in supplement form for its ability to sustain health and assist in cellular repair processes. This essential mineral is frequently included in health regimens aimed at bolstering the body's natural defenses.

As a vital mineral, magnesium is essential for numerous physiological functions and is widely used in supplements and topical formulations. It is particularly noted for its calming effects on the nervous system and its ability to relax muscles, thereby promoting overall physical well-being and stress relief.

Titanium is occasionally employed in therapeutic jewelry due to its lightweight properties and hypoallergenic nature. It is believed to contribute to energy balance in the body, making it beneficial in practices focused on promoting well-being and enhancing physical vitality.

Although less frequently referenced than other metals, platinum is known for its luxurious attributes and is thought to possess calming effects on the body. It may be utilized in holistic health applications to foster tranquility and emotional balance.

In addition to high-frequency metals, another high-frequency material that may be used are certain crystals. In some embodiments, crystals are used in the construction of the furniture. Amethyst and quartz are high-frequency crystals and believed to emit specific frequencies that promote energy balance and alignment within the body. Different crystals have different effects on different portions of the body and various examples of crystals being used in furniture are provided herein. Research suggests that certain crystals possess piezoelectric properties, meaning they can convert mechanical stress into electromagnetic energy, potentially aiding in harmonizing the body's energy fields. Incorporating crystals into furniture design may help create an environment conducive to relaxation, stress reduction and overall well-being.

The choice of fabric within the furniture design is also an important aspect in preferred embodiments. Using high-frequency fabrics in furniture contributes to a more sustainable and healthy living environment, supports overall well-being, enhances comfort, and helps regulate indoor air quality. High-frequency fabrics also promote a sense of serenity through their breathable and hypoallergenic properties, making them suitable for various living spaces and personal preferences. Fabrics that are considered high-frequency include cotton, silk, linen, wool, bamboo, hemp, and Tencel (Lyocell). Furthermore, by choosing natural and sustainable materials for furniture, individuals can create a more harmonious home that fosters a connection to nature and supports eco-friendliness.

One of the most notable high-frequency fabrics is cotton. Renowned for its breathability and moisture absorbent qualities, cotton provides exceptional comfort, making it an ideal choice for furniture upholstery. Its natural hypoallergenic properties cater to sensitive individuals, and the fabric softens with every wash, enhancing the overall comfort and relaxation of any living space.

Silk is another luxurious fabric that adds an elegant touch to furniture design. Its smooth and soft texture not only evokes a sense of opulence but also contributes to a calming atmosphere. Silk naturally regulates temperature, ensuring comfort in various conditions, while its amino acids and proteins can also be beneficial for skin health, making it a unique choice for upholstered items.

Linen is well-loved for its durability and lightness. This fabric is highly breathable and effectively wicks moisture, making it perfect for warm climates. Linen's natural fibers possess antibacterial properties, promoting a healthier living environment. Its timeless aesthetic adds character and sophistication to any room's decor.

Wool is often sought after for its excellent insulating properties. Providing warmth without overheating, wool is a fantastic option for upholstery in colder climates. Additionally, its moisture-wicking abilities contribute to maintaining a dry and comfortable space. The natural elasticity of wool ensures that furniture retains its shape while providing necessary support.

The rise of eco-friendly options has brought bamboo to the forefront of sustainable furniture fabrics. Bamboo fibers are soft and breathable, making them an excellent choice for upholstery. The antimicrobial and hypoallergenic properties of bamboo enhance user comfort, while its sustainability roots promote an eco-conscious lifestyle.

Hemp fabric is increasing in popularity due to its remarkable durability and strength, which makes it resistant to wear and tear. This fabric also offers natural UV resistance, protecting both the fabric and the skin from sun exposure. With its breathable qualities, hemp contributes to comfort and longevity in indoor furnishings.

Lastly, Tencel (Lyocell) is a newer fabric that has gained recognition for its sustainability and luxurious feel. Made from sustainably sourced wood pulp, Tencel is highly breathable and moisture-wicking, helping maintain a fresh and comfortable environment. Its softness and gentle touch make it an excellent addition to any furniture, enhancing both style and comfort.

Certain fabrics can promote comfort and emotional wellness. The choice of fabrics can significantly impact emotional well-being and comfort levels. Natural fabrics such as cotton, linen, and wool are considered high-frequency materials and are preferred for their breathability and tactile properties and are believed to emit higher frequencies. Incorporating soft, natural fabrics into furniture design can enhance the overall sensory experience, promoting relaxation and emotional balance.

However, it has been found that when wool and linen are mixed together that their frequencies cancel each other out. Accordingly, wool and linen should not be used together in a single piece of furniture. Moreover, synthetic materials such as polyester and nylon are not high-frequency materials and resonate at 15 hz to 0 hz such that these fibers should never be used as they are destructive to the body. In contrast, both organic cotton, which resonates at 100 hz and cotton, which resonates at 70 hz, are considered high-frequency materials.

Another high-frequency material is wood. Studies have shown that exposure to natural materials like wood can lower sympathetic nervous system activity, reducing stress levels and promoting relaxation. Furniture crafted from wood such as pine can enhance indoor air quality by regulating humidity levels and minimizing the presence of airborne toxins, fostering a healthier living environment.

Wood may be used in the construction of the furniture designs herein. In addition to wood's high-frequency properties, wood offers both strength and aesthetic appeal. Various parts of furniture utilize wood in distinct ways to enhance functionality and design.

At the core of many furniture pieces is the frame. The frame may be made of wood to provide stability and support. Of course, the frame may also be made from a high-frequency metal. This structural framework is crucial for items like sofas, beds, and chairs, where durability is a must. Complementing the frame, legs play a significant role in the structure of furniture and may be crafted from wood or a high-frequency metal. The legs of tables, chairs, and cabinets not only support the weight of the furniture but also contribute to its overall visual appeal.

The tabletops of dining tables, coffee tables, and desks are often constructed from wood, providing a durable and attractive surface for everyday use. In addition, shelves made from wood are popular for bookcases, cabinets, and wall units, offering sturdy storage solutions that are both functional and visually pleasing. In other embodiments, the tabletops and shelves may also be construct from a high-frequency metal without departing from the teachings herein.

Seating furniture features backrests that may be made of wood or a high-frequency metal, adding style to chairs and benches. Moreover, arms on chairs and sofas may be crafted from wood or a high-frequency metal, further enhancing the aesthetic and providing support for users. When it comes to storage, drawer construction commonly incorporates wood, ensuring that drawers are both functional and long-lasting.

In cabinetry, cabinet doors are frequently made from wood, adding character and elegance to various storage solutions, including armoires and kitchen cabinets. To ensure structural integrity, supports and braces made of wood provide additional stability, especially in larger furniture items like tables and cabinets. In yet other embodiments, a high-frequency metal may be used to make the cabinet doors, supports or braces.

Bed frames may be made from wood, providing durability and a solid foundation for a good night's sleep. In other embodiments, high-frequency metal may be used for the bed frame.

In addition to structural elements, both wood and high-frequency metals may be utilized for decorative elements in furniture design. From moldings to inlays and intricate carvings, these details can significantly enhance the visual interest and unique character of each piece.

Through these various applications, it is clear that wood serves not only as a structural element but also as a key component in the aesthetic and functional qualities of furniture, making it a timeless choice for designers and craftsmen alike.

In preferred embodiments, one or more high-frequency materials such as copper, crystals, natural fabrics and wood are combined in a piece of furniture. One skilled in the art will appreciate from the teachings herein that any combination of copper, crystals, natural fabrics and wood may be used and, in some embodiments, one or more of those materials may be used in combination or individually. In addition, each material copper, crystals, natural fabrics and wood can be used in various different quantities depending on the size of the piece of furniture. As an example, copper and crystals may be used in one piece of furniture while copper, crystal and wood may be used in another. In yet another embodiment, all four of copper crystal, wood and natural fabrics may be used.

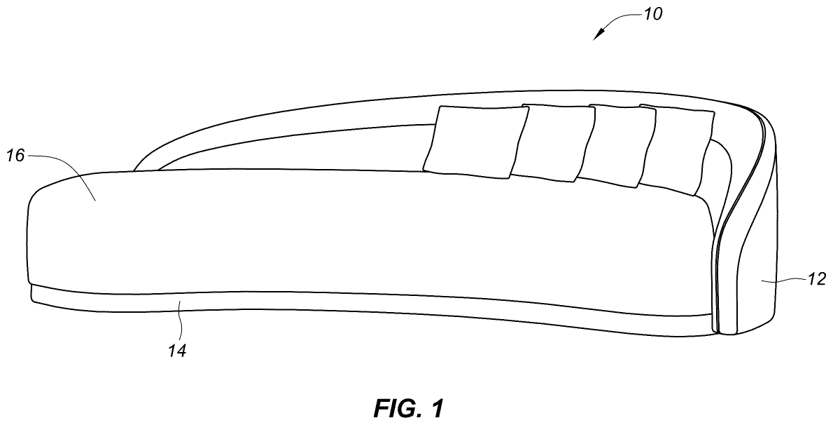

FIG. 1 illustrates an embodiment of a holistic and therapeutic sofa 10 designed using the principles taught herein. In preferred embodiments, sofa 10 includes 15 to 30 pounds of copper. As may be appreciated from FIG. 1, sofa 10 includes a copper back 12 and a copper toe kick 14. However, in other embodiments, sofa 10 may include copper in other locations and may or may not have a copper back 12 and/or copper toe kick 14.

In preferred embodiments, sofa 10 may include between 0.5 lbs. and 2 lbs. of copper balls (not shown in FIG. 1) located inside the sofa 10. In more preferred embodiments, sofa 10 includes 1 lb. of copper balls located inside the sofa 10. In preferred embodiments, the copper balls are located within the sofa 10 near where the heart and crown Chakra centers of the body would be located when a user is sitting or lying on the sofa 10. In yet other embodiments, the copper balls may be placed within the sofa 10 in other locations to align with other Chakra locations when a user is sitting or lying on the sofa 10.

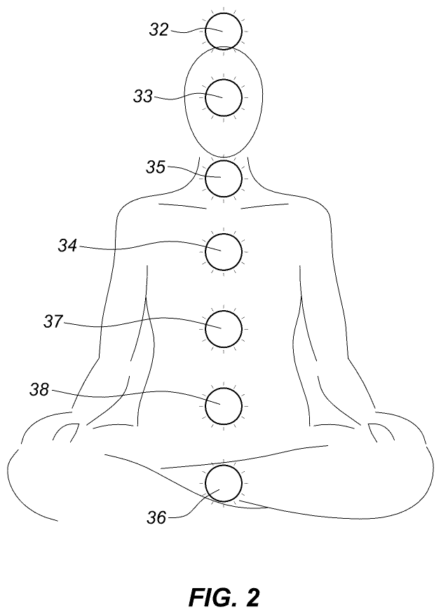

FIG. 2 illustrates the location of the various Chakra known in the human body. The present invention relates to the integration of crystals within furniture designs to enhance the alignment and balance of chakras. This innovative approach aims to incorporate the natural healing properties of crystals into everyday furniture, promoting wellness, tranquility, and positive energy in living spaces.

Looking at FIG. 2, the human body has seven primary chakras, which are energy centers that influence our physical, emotional, and spiritual well-being. The Root Chakra 36, located at the base of the spine, is associated with grounding and stability. The Sacral Chakra 38, situated in the lower abdomen, governs creativity and emotional connection. The Solar Plexus Chakra 37, located in the upper abdomen, is linked to personal power and self-esteem. The Heart Chakra 34, at the center of the chest, represents love and compassion. The Throat Chakra 35, found in the throat area, facilitates communication and self-expression. The Third Eye Chakra 33, located between the eyebrows, is associated with intuition and insight. Finally, the Crown Chakra 32, situated at the top of the head, connects us to higher consciousness and spiritual awareness.

The placement of crystals associated with each chakra within furniture aims to harmonize energy flow and improve the overall well-being of individuals using the furniture.

Some non-limiting examples of furniture designs incorporating the high-frequency materials are presented below. As one will appreciate, other designs for furniture may be created without departing from the scope of the teachings herein.

For the Base Chakra 36 (Root Chakra), linked with grounding and stability, high-frequency crystals can be strategically integrated into furniture made from high-frequency materials. For instance, storage ottomans crafted from sustainably sourced hardwood can feature hidden compartments with removable bottom panels that hold high-frequency crystals. In some embodiments, the compartments may be lined with natural cotton fabric to enhance energy flow. The use of natural dyes to color the fabric can further promote a harmonious atmosphere. Additionally, wooden bed frames made from reclaimed wood can be designed with hollow spaces in the legs, providing secure compartments for high frequency crystals, easily accessible through discreet openings underneath.

Although in various embodiments any high-frequency crystals may be used, in preferred embodiment, Red Jasper and Hematite may be used. Red Jasper and Hematite are particularly well known to be effective with the Base Chakra 36.

When addressing the Sacral Chakra 38, associated with creativity and emotional balance, furniture designs can benefit from multiple applications. A round dining table made from quality hardwood with a natural finish can incorporate an inner ring just beneath the tabletop to hold larger high-frequency crystal pieces. In preferred embodiments, the high-frequency crystals may be covered by a glass surface that allows for visibility.

In yet other embodiments, couches upholstered in organic linen fabric can enhance this energy by having snug compartments on the armrests for polished high frequency crystals, complete with cushioned wool liners and magnetic closures for easy access.

In targeting the Solar Plexus Chakra 37, which is linked to personal power and self-esteem, furniture such as coffee tables and bookshelves can be transformed to accommodate high-frequency crystals. In some embodiments, Citrine and Yellow Jasper may be used. In yet other embodiments, a coffee table constructed from a natural wood or high-frequency metal can feature a lower shelf equipped with a sliding drawer lined in natural wool to hold high-frequency crystals. Bookshelves made from natural wood can include illuminated sections behind frosted glass doors to display high-frequency crystals.

The Heart Chakra 34, associated with love and compassion, can be supported through furniture like accent chairs and bedside tables made from organic materials. Accent chairs crafted from sustainable timber can incorporate pockets that are designed to hold high-frequency crystals such as Rose Quartz pieces. Similarly, bedside tables made from natural wood or high-frequency metals can feature small drawers specifically for high-frequency crystals, allowing users to select different crystals based on their emotional needs each night.

To facilitate communication and self-expression associated with the Throat Chakram 35, furniture designs for desks and bookshelves can include integrated crystal holders made of natural materials. A workstation made of polished hardwood may feature a recessed area within the desktop, lined with cotton velvet to securely hold high-frequency crystals such as Aquamarine or Blue Lace Agate crystals, complete with a decorative copper cover that doubles as a paperweight. Bookshelves constructed from natural wood can feature eye-level transparent compartments with glass fronts housing high-frequency crystals.

The Third Eye Chakra 33, connected to intuition and insight, can be supported through recliners and floor lamps made with high-frequency materials. Recliners made of sustainably harvested wood can be designed with cushioned headrests containing secure pockets for high-frequency crystals.

Floor lamps can feature bases made from natural wood with integrated holders, allowing light to glow through the crystals, illuminating the surrounding area and serving as a focal point for meditation.

The crown chakra 32 is believed to promote overall health and well-being on physical, emotional, mental, and spiritual levels. For the Crown Chakra 32, which connects to higher consciousness, furniture pieces such as sofas and recliners with headrests can be designed to incorporate high frequency crystals within the headrests. Sofas made from a framework of durable hardwood can include headrests with cushioned pockets specifically tailored for high-frequency crystals.

Within any piece of furniture, especially sofas, loveseats and recliners, compartments or pockets can be made to hold copper balls, crystals or other high-frequency enhancing materials. One skilled in the art of furniture making, will appreciate where within sofa 10 the copper balls could be placed to align with the crown Chakra 32 and the heart Chakra 34 or any other Chakra. In particular, copper is known to resonate with the root Chakra 36 and sacral Chakra 38.

Copper is believed to enhance the energy conductivity of crystals, allowing for a stronger flow of energy. The combination of copper and crystals creates a synergistic effect, magnifying the benefits and promoting balance and well-being. By incorporating copper into holistic wellness practices, individuals can align their Chakras, amplify energy flow, and enhance the benefits of crystals, ultimately promoting harmony and well-being in body, mind, and spirit.

In preferred embodiments, sofa 10 includes between 1 and 10 lbs of crystals (not shown in FIG. 1). Crystals aid in balancing all the chakras. In yet more preferred embodiments, sofa 10 may include 3 lbs. to 4 lbs. of crystals. In some embodiments, the sofa 10 may include 3 lbs. to 4 lbs. of amethyst and rose quartz crystals in combination. Amethyst and rose quartz crystals vibrate at 852 hz. In yet other embodiments, the sofa 10 may include 3 lbs. to 4 lbs. of clear quartz crystals which vibrate at 963 hz.

In yet other embodiments, Citrine (“Sun Stone”) may be used as the high-frequency crystal.

The crystals may be amplified by the pure copper balls. The copper amplification can double the energy of the crystals.

In preferred embodiments, a high frequency material 16 is used for the upholstery of the sofa 10. In preferred embodiments, the high frequency material 16 is wool. Pure wool resonates at a tremendous 5000 hz—(50 times the natural frequency of the human body!). Thus, the healing and positive potential of wool on the body is exponentially influencing our energy and health. In the embodiment shown in FIG. 1, 54 yards of wool upholstery is used. In other embodiments, more or less wool may be used depending on the size of the piece of furniture.

In some embodiments, copper fabric may be included. Copper fabric can amplify the wool frequency of 5000 hz to increase exposure of the body to even higher frequencies. In preferred embodiments, between 1 and 20 yards of copper fabric are used. In more preferred embodiments, between 5 and 15 yards of copper fabric are used. In yet even more preferred embodiments, between 9 and 11 yards of copper fabric are used. In the embodiment shown in FIG. 1, 10 yards of coper fabric is used.

The sofa 10 shown in FIG. 1 exposes the user to 963 hz+852 hz+5000 hz=6,815 hz plus exposure to unique frequencies influencing our energy and health to give over 100 hz exposure to high frequency.

As mentioned above, wood is preferably used in the sofa 10. In preferred embodiments, Pine is used as the wood. Pine is the tree of Peace, and a Symbol of Purification & Cleansing. Pine contains chemicals that might have activity against pain and swelling (inflammation). It also seems to be mildly effective in killing bacteria and fungus.

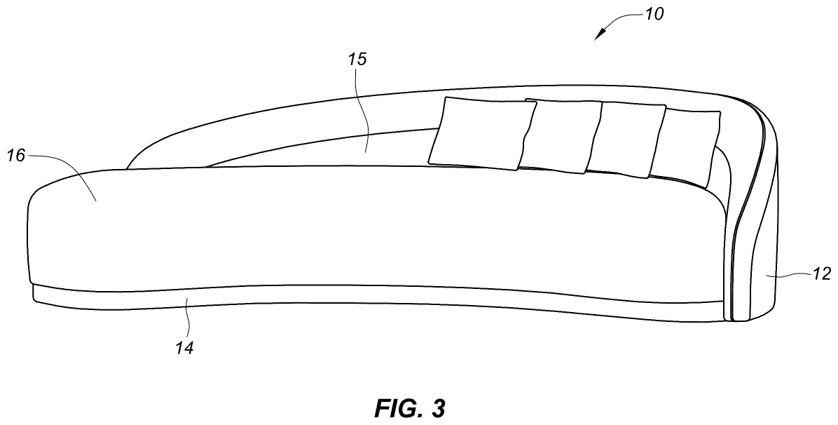

FIG. 3 illustrates an embodiment of a holistic and therapeutic sofa similar to the one in FIG. 1 but with an additional copper back 15.

In preferred embodiments, the furniture, like the sofa shown in FIGS. 1 and 3 for example, consists of: 1.) Pine wood frames; 2.) copper fabric backing pockets with foiled gemstones; and 3.) softened edges consistent with sacred geometry. In some embodiments, the furniture may also include copper mesh fabric pockets to be filled with gemstones/crystals. In preferred embodiments, the gemstones/crystals may be foiled in copper to amplify the frequency flow into the body, enhancing energy flow. In some embodiments, the clients may select the crystals/gemstones to be filed from the group consisting of amethyst, Citrine, clear quartz and rose quartz.

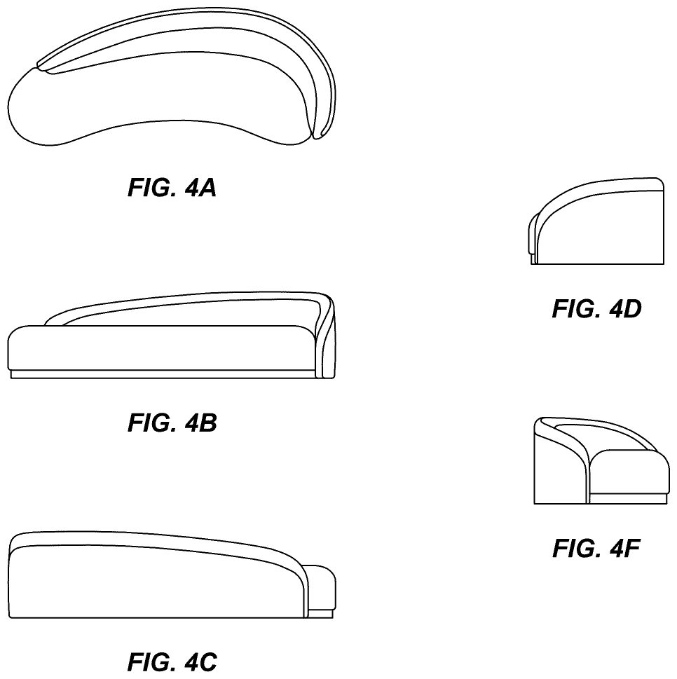

FIGS. 4A-4F illustrate various views of the sofa of FIGS. 1 and 3 with some dimensions for reference. As one skilled in the art will appreciate, these dimensions are just an example of one embodiment and other embodiments may have different dimensions.

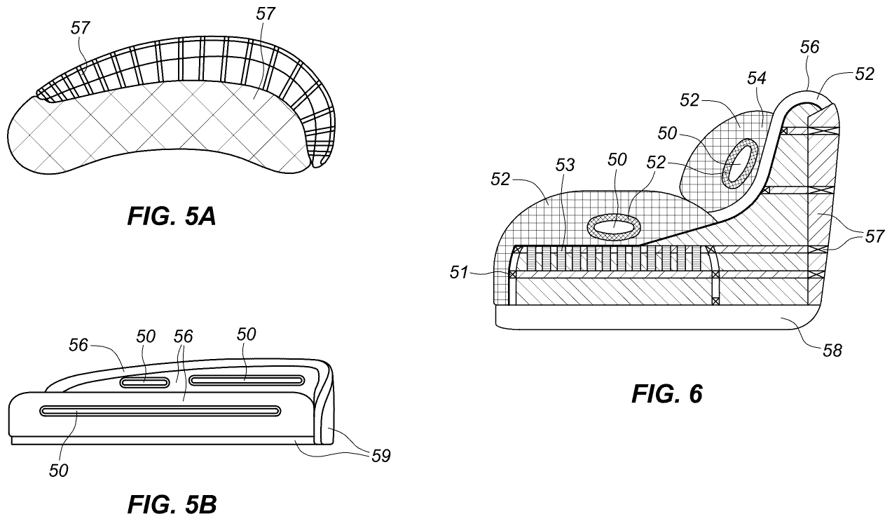

FIG. 5A-5C illustrates various interior views of the sofa of FIGS. 1, 3 and 4. FIG. 6 illustrates an internal view of one embodiment of a sofa made according to the teachings herein.

As may be seen in FIGS. 5A-5C and 6, crystals may be inserted inside inserts 50 with silk envelopes wrapped in copper mesh fabric. As may be seen, the crystals 50 may extend down the longitudinal length of the sofa 10 in various different locations. The crystals and pure copper balls wrapped in copper mesh fabric envelopes encased in silk. The crystals may be in both the back portion of the seat portion of the sofa or both.

In the embodiment of a sofa shown in FIGS. 5A-5C, the sofa is made with a certified sustainable green pine wood frame 57 and has a copper mesh fabric backing 51. The pine wood frame 57 included both horizontal and vertical members.

The sofa is filled with a sustainable batting 52 in numerous locations. In preferred embodiments, the batting is made from a high-frequency natural material like wool but in other embodiments, the batting made from a material that is non-high-frequency. In preferred embodiments, the batting 52 has a copper mesh fabric.

The sofa has spring metal coils 53. Although preferably made from a high frequency metal, the spring metal coils 53 may be made from a non-high-frequency metal without departing from the scope of furniture consisting essential of high-frequency materials as taught herein.

The upholstery fabric 56 is customizable and has a copper mesh fabric backing.

The sofa features inserts with crystals and pure copper balls 50 wrapped in copper mesh fabric envelopes encased in silk. The sofa has decorative zippers 54 that allow you to change the crystal inserts 50 inside.

The sofa has a certified sustainable green pine wood base 58 wrapped with 22 gauge copper. The copper 59 may be used on the back and base in a decorative way.

One aspect of the inventions taught herein lies as much in the absence of certain materials as much as it does in their presence. For example, the present application contemplates furniture made entirely of high frequency materials such as high frequency materials with the absence of low frequency materials where higher frequency materials exist. For example, furniture may be made of the materials discussed herein and no other materials or at least not other materials of any significant quantity. Accordingly, in some embodiments, the sofa 10 is constructed entirely of pine, wool, copper and crystals.

What is claimed is:

1. Furniture consisting essentially of:
a structural member made of wood;
high-frequency metals selected from the group consists of: copper, Silver, Gold, Zinc, Magnesium, Titanium, and Platinum;
upholstery made from a high-frequency fabric selected from the group consisting of: cotton, silk, linen, wool, bamboo, hemp, and Tencel (Lyocell); and
a plurality of high-frequency crystals located in internal compartments within the furniture.

2. The furniture of claim 1 wherein a plurality of copper balls is located in close proximity to the plurality of high frequency crystals.

3. The furniture of claim 1, wherein the structural member is made of pine wood.

4. The furniture of claim 1, wherein the furniture comprises a sofa.

5. The furniture of claim 1, wherein the high-frequency crystals are located in pockets within the furniture upholstery.

6. The furniture of claim 1, wherein the high-frequency crystals are enclosed in copper mesh fabric pockets.

7. The furniture of claim 1, wherein between 1 and 10 pounds of high-frequency crystals are used.

8. The furniture of claim 1, where in the high frequency crystals are selected from the group consisting of: Red Jasper, Hematite, Amethyst, Rose Quartz, Clear Quartz, Citrine, Yellow Jasper and Rose Quartz.

9. The furniture of claim 1, wherein the high frequency crystals are amethyst and rose quartz crystals in combination.

10. The furniture of claim 1, wherein the high-frequency crystals are foiled in copper.

11. A sofa consisting essentially of:
a structural member made of wood;
high-frequency metals selected from the group consists of: copper, Silver, Gold, Zinc, Magnesium, Titanium, and Platinum;
upholstery and batting made from a high-frequency fabric selected from the group consisting of: cotton, silk, linen, wool, bamboo, hemp, and Tencel (Lyocell); and
a plurality of high-frequency crystals located in internal compartments within the furniture.

12. The furniture of claim 1, wherein a plurality of copper balls is located in close proximity to the plurality of high frequency crystals.

13. The furniture of claim 1, wherein the structural member is made of pine wood.

14. The furniture of claim 1, wherein the high-frequency crystals are located in compartments within the furniture upholstery.

15. The furniture of claim 1, wherein the high-frequency crystals are enclosed in copper mesh fabric pockets.

16. The furniture of claim 1, wherein between 1 and 10 pounds of high-frequency crystals are used.

17. The furniture of claim 1, where in the high frequency crystals are selected from the group consisting of: Red Jasper, Hematite, Amethyst, Rose Quartz, Clear Quartz, Citrine, Yellow Jasper and Rose Quartz.

18. The furniture of claim 1, wherein the high frequency crystals are amethyst and rose quartz crystals in combination.

19. The furniture of claim 1, wherein the high-frequency crystals are foiled in copper.

20. A sofa consisting essentially of:
a structural frame made of pine wood;
high-frequency metals selected from the group consists of: copper, Silver, Gold, Zinc, Magnesium, Titanium, and Platinum;
upholstery and batting made from a high-frequency fabric selected from the group consisting of: cotton, silk, linen, wool, bamboo, hemp, and Tencel (Lyocell);
a plurality of high-frequency crystals located in internal compartments within the furniture wherein the high-frequency crystals are selected from the group consisting of: Red Jasper, Hematite, Amethyst, Rose Quartz, Clear Quartz, Citrine, Yellow Jasper and Rose Quartz; and
a plurality of copper balls located in close proximity to the plurality of high frequency crystals.
